(19) United States
(12) Patent Application Publication
(10) Pub. No.: US 2016/0346480 A1
(43) Pub. Date: Dec. 1, 2016

(54) SYRINGES AND METHODS OF USING THEM TO TREAT OVERACTIVE BLADDER

(71) Applicants: Jonathan L. Gleason, Roanoke, VA (US); William J. Greer, Roanoke, VA (US)

(72) Inventors: Jonathan L. Gleason, Roanoke, VA (US); William J. Greer, Roanoke, VA (US)

(21) Appl. No.: 15/154,992

(22) Filed: May 14, 2016

Related U.S. Application Data
(60) Provisional application No. 62/162,227, filed on May 15, 2015.

(51) Int. Cl. A61M 5/315; A61K 9/00; A61K 38/48; A61B 1/307; A61K 9/19

(52) U.S. Cl. CPC A61M 5/31593; A61B 1/307; A61K 9/19; A61K 38/4893; C12Y 304/24069; A61K 9/0034; A61M 2205/582; A61M 2205/502; A61M 2205/52

(57) ABSTRACT

Certain configurations are directed to a syringe configured to provide tactile feedback and that permits injection of a plurality of individual metered volumes. In some embodiments, the syringe can be used to inject a metered volume of a botulinum toxin at each of a plurality of separate sites of a bladder wall to treat overactive bladder.

### PRIORITY APPLICATION

This application is related to, and claims priority to and the benefit of, U.S. Patent Application No. 62/162,227 filed on May 15, 2015, the entire disclosure of which is hereby incorporated herein by reference for all purposes.

### TECHNOLOGICAL FIELD

This application is related to syringes and methods of using them to treat overactive bladder. More particularly, certain embodiments are directed to syringes and their use to inject a botulinum toxin into a plurality of individual sites of a bladder wall to treat overactive bladder.

### BACKGROUND

Overactive bladder is characterized by a sudden urge to urinate. The urge may lead to involuntary urination. Overactive bladder is common in older adults particularly in women over the age of forty five.

### SUMMARY

Certain configurations of a syringe are described herein that can provide tactile feedback and be used in treating overactive bladder. In certain instances, the syringe can be used to provide a plurality of individual injections each of a metered volume without a user being able to view the syringe during the injections.

In one aspect, a method of treating overactive bladder comprising injecting a bladder wall at a plurality of individual sites with a botulinum toxin, e.g., onabotulinumtoxinA, from a syringe configured with a plurality of individual internal stops to dispense a defined volume from the syringe at each injection site as a plunger of the syringe is depressed from a first internal stop to a second internal stop to treat the overactive bladder is provided.

In certain embodiments, the method comprises injecting the botulinum toxin in a m×n array into the bladder wall. In other embodiments, m of the array is two to thirty and n of the array is two to thirty. In some instances, the method may comprise configuring the syringe to provide tactile feedback at each of the plurality of individual internal stops. In further examples, the method may comprise configuring the syringe to provide substantially no noise as the plunger is depressed from the first stop to the second stop. In certain embodiments, the method comprises configuring the syringe with a total volume to permit injection into at least fifteen or at least twenty individual sites in the bladder wall. In some examples, the method comprises configuring a barrel of the syringe with an interior ridge that engages a projection on a plunger of the syringe at the first internal stop. In certain examples, the method comprises loading the syringe with lyophilized botulinum toxin. In additional examples, the method comprises solubilizing the lyophilized botulinum toxin by introducing a liquid into the syringe with the lyophilized botulinum toxin. In some examples, the method comprises selecting the botulinum toxin to be onabotulinumtoxinA. In further embodiments, the method comprises positioning the syringe to inject substantially the same amount of botulinum toxin at each of the plurality of individual sites in the bladder wall when a user injecting the botulinum toxin cannot view the syringe. In some examples, the method comprises using the syringe with a cystoscope to inject the botulinum toxin. In other examples, the method comprises using a device configured to count the number of injections into the bladder wall. In some instances, the method comprises using a device configured with a processor during the injection process. In additional examples, the method comprises configuring the processor to display a grid on an image provided by the cystoscope. In some embodiments, the method comprises configuring the grid with an indicator site of each of the plurality of injection sites and removing the indicator once the syringe has been used to inject the botulinum toxin into the bladder wall at a particular indicator site. In additional examples, the method comprises using the syringe to inject at least twenty individual sites. For example, by using a tactile feedback syringe, about 20 individual sites can be injected in 10-25% less time than when using a non-tactile feedback syringe. In certain configurations, the method comprises configuring a plunger of the syringe with a plurality of radial projections and configuring a barrel of the syringe with a plurality of interior ridges configured to provide tactile feedback when at least one of the radial projections contacts at least one of the interior ridges. In some embodiments, the method comprises configuring the plunger to comprise three radial projections spaced about 120 degrees apart and configuring the barrel with at least one circumferential interior ridge, in which depression of the plunger from a first position where the a first projection of the plunger engages the circumferential ridge of the barrel to a second position where a second projection of the plunger engages the circumferential ridge of the barrel dispenses a metered volume of the botulinum toxin from the syringe. In additional examples, the method comprises training a bladder muscle by inducing voiding of the bladder at a selected interval.

In another aspect, a syringe comprising a plunger and a barrel is provided. In certain configurations, the plunger comprises at least two projections extending radially from a longitudinal body. In some examples, the barrel comprises a body configured to receive the plunger and comprising a plurality of circumferential grooves each positioned longitudinally along the body of the barrel, wherein each circumferential groove in a first radial plane is configured to engage one of the at least two projections at a first position of the plunger, in which an adjacent circumferential groove in a second radial plane is configured to engage one of the at least two projections in a second position of the plunger, in which movement of the radial projections of the plunger from the first radial plane to the second radial plane provides tactile feedback and is configured to dispense a metered volume of fluid.

In certain embodiments, movement of the projections of the plunger from the first radial plane to the second radial plane provides the tactile feedback without providing any audible feedback, e.g., the patient cannot hear any audible clicking or noise during depression of the plunger and/or the operator of the syringe cannot hear any audible clicking or noise during depression of the plunger at a typical use distance for a particular procedure, for example, treating overactive bladder. In other embodiments, the plunger comprises two radial projections at each of a plurality of different radial planes along the longitudinal body of the plunger. In some instances, the barrel comprises a corresponding number of circumferential grooves positioned in a plurality of different radial planes along the body of the barrel. In certain examples, the plunger comprises three substantially equal projections extending radially from the longitudinal body of the plunger and spaced about 120 degrees from each other. In some embodiments, the barrel comprises three circumferential grooves in each of a plurality of radial planes of the body, in which the plurality of radial planes are spaced apart from each other an effective distance to provide the metered volume of fluid when the three projections of the plunger are moved from the first radial plane to the second radial plane. In certain examples, the syringe comprises an internal stop at a base of the barrel, the internal stop configured to engage a terminal end of the plunger to prevent further movement of the plunger. In other examples, each of the three substantially equal projections of the plunger is positioned orthogonal to the longitudinal body of the plunger. In certain examples, the plunger comprises with three radial projections spaced about 120 degrees from each other at a plurality of different radial planes along the longitudinal body of the plunger. In some embodiments, the barrel comprises two or three circumferential grooves at each radial plane, in which the body of the barrel comprises substantially the same number of radial planes with circumferential grooves as the number of radial planes comprising radial projections present along the longitudinal body of the plunger. In some examples, the plunger and the barrel each comprise materials, e.g., glass, high temperatures plastics, medical grade plastics, etc., capable of withstanding autoclaving without deforming the projection and the ridges. In some embodiments, the barrel and the plunger (and any features thereon) may be molded to provide a desired configuration. In certain embodiments, the syringe comprises an internal stop along the body of the barrel, the internal stop configured to prevent depression of the plunger further once the plunger contacts the internal stop. In some examples, the syringe comprises at least one volume indicator on an outside surface of the barrel. In certain examples, the barrel comprises a coupling configured to couple the syringe to a needle. In other examples, at least one of the circumferential grooves is configured to permit one way movement of the plunger in the barrel. In other examples, each of the circumferential grooves is configured to permit one way movement of the plunger in the barrel. In some embodiments, at least one of the radial projections of the plunger is configured to permit one way movement of the plunger in the barrel. In certain embodiments, each of the radial projections of the plunger is configured to permit one way movement of the plunger in the barrel. In certain examples, the syringe is sized and arranged to dispense about ten to about twenty individual metered volumes of fluid or about fifteen to about twenty individual metered volumes of fluid.

In another aspect, a syringe comprising a plunger comprising at least one projection extending radially from a longitudinal body, and a barrel comprising a body configured to receive the plunger and comprising at least one circumferential ridge positioned longitudinally along the body of the barrel, wherein the circumferential ridge is positioned in a first radial plane and is configured to engage the at least one projection of the plunger at a first position of the plunger to provide tactile feedback and to dispense a metered volume of fluid.

In certain embodiments, the plunger comprises a first radial projection and a second radial projection. In other embodiments, movement of the first radial projection of the plunger from the first radial plane to a second radial below the first radial plane to permit engagement of the second radial projection with the circumferential ridge provides the tactile feedback without providing any audible feedback. In some configurations, the plunger comprises with two radial projections at each of a plurality of different radial planes along the longitudinal body of the plunger. In other configurations, the barrel comprises a second circumferential ridge position in a radial plane different that the radial plane of the circumferential ridge corresponding number of circumferential ridges positioned in a plurality of different radial planes along the body of the barrel. In some examples, the plunger comprises three substantially equal projections in a single radial plane that extend radially from the longitudinal body of the plunger and spaced about 120 degrees from each other. In other examples, the barrel comprises three circumferential ridges in each of a plurality of different radial planes of the body, in which the plurality of different radial planes are spaced apart from each other an effective distance to provide the metered volume of fluid when the three radial projections of the plunger are moved from the first radial plane to the second radial plane. In some embodiments, the syringe comprises an internal stop at a base of the barrel, the internal stop configured to engage a terminal end of the plunger to prevent further movement of the plunger. In other embodiments, each of the three substantially equal radial projections of the plunger is positioned orthogonal to the longitudinal body of the plunger. In other examples, the plunger comprises three radial projections spaced about 120 degrees from each other at each of a plurality of different radial planes along the longitudinal body of the plunger. In some instances, the barrel comprises only one circumferential ridges at the first radial plane. In other examples, the plunger and the barrel each comprise materials capable of withstanding autoclaving without deforming the projection and the ridges. In certain examples, the syringe comprises an internal stop along the body of the barrel, the internal stop configured to prevent depression of the plunger further once the plunger contacts the internal stop. In additional examples, the syringe comprises at least one volume indicator on an outside surface of the barrel. In certain embodiments, the barrel comprises a coupling configured to couple the syringe to a needle. In other embodiments, the circumferential ridge of the barrel is configured to permit one way movement of the plunger in the barrel. In additional examples, the barrel and the plunger each comprise a material selected from the group consisting of a plastic, a glass and combinations thereof. In some configurations, at least one of the radial projection of the plunger is configured to permit one way movement of the plunger in the barrel. In other configurations, the syringe comprises only one circumferential ridge on the barrel and the plunger comprises a radial projection in each of plurality of different radial planes, in which the plunger comprises enough radial projections to dispense about twenty individual metered volumes of fluid with tactile feedback during all dispensed volumes. In some examples, the syringe is sized and arranged to dispense about ten to about twenty individual metered volumes of fluid or about fifteen to about twenty individual metered volumes of fluid.

In an additional aspect, a kit comprising one of the syringes described herein, a lyophilized botulinum toxin, and instructions for using the syringe and the lyophilized botulinum toxin to treat overactive bladder is disclosed.

In certain embodiments, the kit comprises an injection solution effective to solubilize the lyophilized botulinum toxin. In other instances, the kit comprises a needle configured to couple to the syringe. In some examples, the kit comprises a fluid line configured to couple to the needle and to the syringe. In additional examples, the lyophilized botulinum toxin of the kit is lyophilized onabotulinumtoxinA.

In another aspect, a kit comprising one of the syringe described herein that comprises a lyophilized botulinum toxin, and instructions for using the syringe comprising the lyophilized botulinum toxin to treat overactive bladder is provided.

In certain embodiments, the kit comprises an injection solution effective to solubilize the lyophilized botulinum toxin. In other embodiments, the kit comprises a needle configured to couple to the syringe. In some examples, the kit comprises a fluid line configured to couple to the needle and to the syringe. In additional examples, the lyophilized botulinum toxin of the kit is lyophilized onabotulinumtoxinA.

In an additional aspect, a method of treating overactive bladder comprising providing a syringe as described herein, and providing instructions for using the syringe with a botulinum toxin to treat overactive bladder is provided.

In certain examples, the method comprises providing the botulinum toxin. In other examples, the method comprises providing a needle configured for use with the syringe. In further examples, the method comprises providing a training device configured to retrain the treated overactive bladder. In additional examples, the method comprises providing instructions for retraining the treated overactive bladder.

Additional features, aspect, examples, configurations and embodiments are described in more detail below.

### BRIEF DESCRIPTION OF THE FIGURES

Certain embodiments are described with reference to the accompanying figures in which:

FIG. 1A is an illustration of a syringe barrel, in accordance with certain examples;

FIG. 1B is an illustration of a syringe plunger, in accordance with certain examples;

FIG. 2A is an illustration of a portion of a syringe barrel, in accordance with certain embodiments;

FIG. 2B is an illustration of a portion of a syringe plunger, in accordance with certain embodiments;

FIG. 3 is an illustration of a syringe barrel comprising a plurality of circumferential grooves, in accordance with certain examples;

FIG. 4A is an illustration of a syringe plunger comprising two radial projections in a single radial plane, in accordance with certain embodiments;

FIG. 4B is an illustration of a syringe barrel comprising circumferential grooves in a plurality of radial planes, in accordance with certain examples;

FIG. 5 is top view of a plunger showing three radial projections spaced about 120 degrees from each other, in accordance with certain configurations;

FIG. 6A is a barrel comprising a single set of circumferential grooves in one radial plane, in accordance with certain examples;

FIG. 6B is a plunger comprising sets of radial projections in a plurality of radial planes, in accordance with certain examples;

FIG. 7A is a barrel comprising two sets of circumferential grooves in different radial planes, in accordance with certain examples;

FIG. 7B is a plunger comprising sets of radial projections in a plurality of radial planes along a longitudinal axis of the plunger, in accordance with certain examples;

FIG. 8A is a barrel comprising sets of radial projections in one, or a plurality, of radial planes along a longitudinal axis of the barrel, in accordance with certain examples;

FIG. 8B is a plunger comprising two sets of circumferential grooves in different radial planes along the longitudinal axis of the plunger, in accordance with certain examples;

FIG. 9A is an illustration of a barrel comprising a circumferential ridge, and FIG. 9B is an illustration of a plunger comprising a plurality of radial projections, in accordance with certain embodiments;

FIG. 10A is an illustration of a barrel comprising two circumferential ridges, and FIG. 10B is an illustration of a plunger comprising a plurality of radial projections, in accordance with certain embodiments;

FIG. 11 is another illustration of a plunger that can be used with the barrels described herein, in accordance with certain configurations;

FIG. 12 is an illustration of a grid showing various injection sites in a bladder wall, in accordance with certain embodiments; and

FIGS. 13A-13C are photographs showing a prototype barrel and plunger, respectively, in accordance with certain examples.

It will be recognized by the person of ordinary skill in the art, given the benefit of this disclosure, that certain dimensions or features in the figures may have been enlarged, distorted or shown in an otherwise unconventional or non-proportional manner to provide a more user friendly version of the figures. No particular length, diameter or wall thickness, is intended by the depictions in the figures, and relative sizes of the figure components are not intended to limit the sizes of any of the components in the figures. Where dimensions or values are specified in the description below, the dimensions or values are provided for illustrative purposes only.

### DETAILED DESCRIPTION

Certain embodiments are described below with reference to singular and plural terms in order to provide a more user friendly description of the technology disclosed herein. These terms are used for convenience purposes only and are not intended to limit the syringes and their use as including or excluding certain features unless otherwise noted as being present in a particular embodiment described herein.

In certain instances, syringes and their methods of use are described below with reference to injection of a toxin. If desired, however, the syringes can be used to inject other materials or agent (either alone or with the toxin) including, for example, anticholinergic medications, oxybutinin, trospium, tolteridone, solifenacin, darifenacin, fesoterodine, mirabegron or other agents. Injection of agents commonly administered through oral routes may provide beneficial results without the drawbacks of the orally administered agents, e.g., no dry mouth.

In certain configurations of the syringes described herein, the syringe or the barrel, or both, can include one or more features that are configured to provide tactile feedback during use of the syringe. For example, one or both of the syringe and the barrel may include one or more features, e.g., a radial projection, a circumferential groove or a circumferential ridge, that is configured to provide tactile feedback to a user as the user depresses the plunger of the syringe. In certain examples, the syringe can include a barrel with a first feature and a plunger with a second feature. The first and second features can interact with each other, e.g., engage each other for some period, to provide tactile feedback to a user that is depressing the plunger. For example, in many instances where a syringe is used, the user may not be able to see any markings on the barrel, e.g., volumetric markings, to be able to determine if a particular volume of fluid is being injected. Over injection may result in too much drug or agent being injected and under injection may result in too little drug or agent being injected. In some instances of the syringes described herein, the first and second features can interact such that tactile feedback is provided to alert the user that a metered volume has been delivered. In some configurations, the features can interact at a first internal stop or area of the barrel to provide for tactile feedback. Depression of the plunger to a second internal stop provides additional feedback alerting the user that a desired volume of fluid has been injected. In certain examples, the desired volume of fluid between the two stops can be about 0.1 cc to about 10 cc, more particularly about 0.5 cc to about 5 cc, e.g., about 1 cc to about 3 cc.

In certain configurations, the syringe components described herein may be described as including ridges, projections or grooves. For clarity, a ridge generally extends away from the body and into another space, e.g., a ridge of a barrel would extend into the interior space designed to receive a plunger. A projection may also extend away from the body of a syringe. A groove typically would extend into the body of the barrel of the syringe, e.g., in a direction away from the interior space of the barrel in the case where a barrel includes a groove.

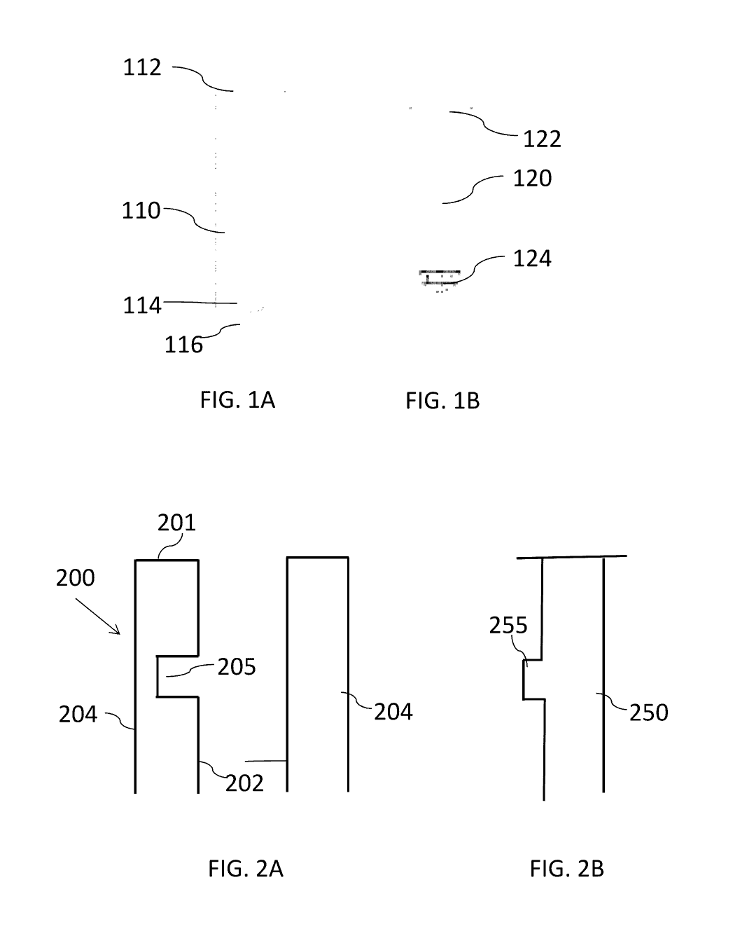

In certain embodiments and referring to FIGS. 1A and 1B, an exploded view of a syringe is shown. The syringe comprises a barrel 110 and a plunger 120. The diameter of the barrel 110 is sized and arranged to permit insertion of the plunger 120 into the barrel 110 at a proximal end 112 and provide for pneumatic and/or hydraulic force by depression of the plunger 110 toward a distal end 114 of the barrel 110. The plunger 120 may comprise a first end 122 that is depressed manually by a user and a second end 124 which can create a generally fluid tight seal to the inner surface of the barrel 120 to force fluid from the end 114 of the barrel 110 during depression of the plunger 120. The barrel 120 generally comprises a coupler 116 that can couple to a needle (not shown) or a fluid line that is coupled to a needle. In use of the syringe to treat overactive bladder, the syringe is used to inject a plurality of different injection sites, e.g., 10-30 or 10-20 or 15-20, with a botulinum toxin or other agent. For example, a plurality of individual injection sites in a bladder wall (as discussed in more detail below) can be injected with a metered volume from the syringe to treat overactive bladder.

In some instances, a user may not be able to view the syringe during injection into the bladder wall. For example, in a typical injection procedure the subject is resting on a table with their feet in stirrups. A user would insert a needle coupled to the syringe into the bladder and adjacent to the bladder wall. For example, a needle can be placed through a cystoscope positioned in the urethra. The insertion process may preclude the user from being able to view the syringe. In particular, during use of a cystoscope (or other viewing device) to permit the user to view the bladder wall, it is not possible to simultaneously view the bladder wall on a video screen electrically coupled to the cystoscope and view any markings on the syringe. The inability to view both devices can reduce the likelihood that the same volume is injected into each of the plurality of injection sites of the bladder wall. To increase precision and reproducibility from injection to injection, the syringe may comprise a feature on the barrel and/or a feature on the plunger that provides feedback to the syringe user that a metered volume of fluid has been injected for a selected travel distance of the plunger. Referring to FIG. 2A, a close up view of a barrel of a syringe is shown. The barrel 200 comprises an inner wall 202 and an outer wall 204. A circumferential groove 205 penetrates into the inner wall 202. The circumferential groove 205 may traverse a desired distance around the body of the syringe, e.g., 45 degrees, 60 degrees, 90 degrees, 120 degrees, 150 degrees, 180 degrees or even up to 360 degrees, and generally resides within a selected radial plane of the barrel 200. As shown in FIG. 2A, the circumferential groove traverses 180 degrees or less. The groove 205 is designed to reversibly engage with a radial projection 255 on a plunger 250 (see FIG. 2B). For example, as the radial projection 255 slides from a first position above the groove 205 and toward a distal end (opposite of the proximal end 201) of the barrel 200, the radial projection 255 can engage the groove 205 which results in the radial projection 255 occupying the space formed by the groove 205. This engagement causes a force to be felt by a user depressing the plunger 250. To overcome the force, the user depresses the plunger 250 with sufficient force to disengage the radial projection 255 from the groove 205. This disengagement causes movement of the radial projection 255 downward toward a distal end (not shown) of the barrel and dispenses additional fluid.

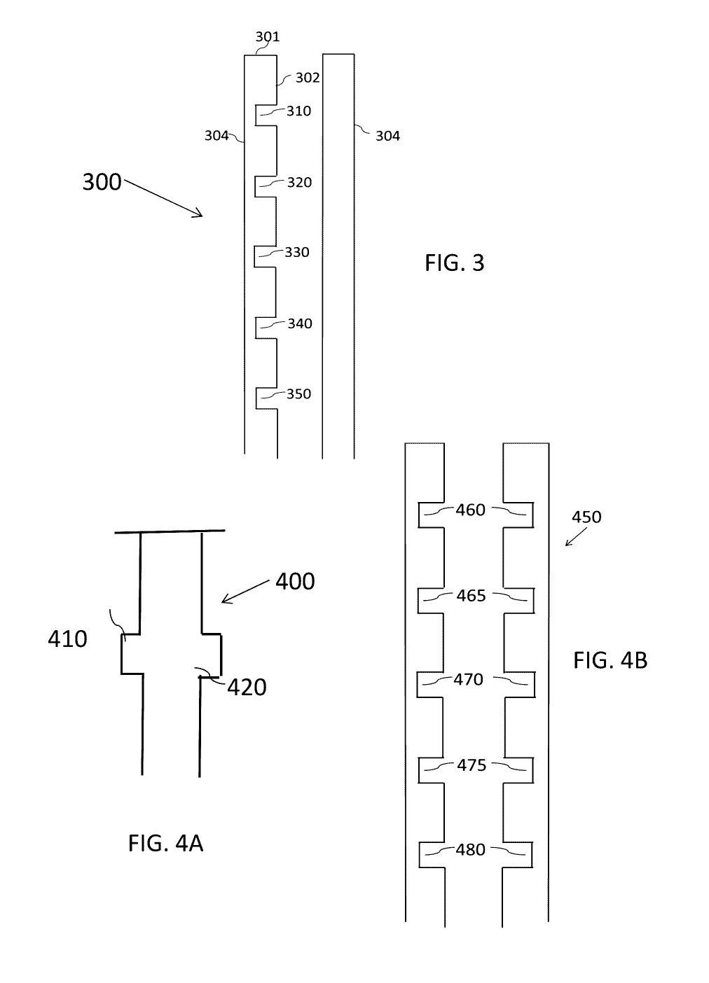

In certain examples, to permit dispensing of a plurality of metered volumes of substantially the same volume, a plurality of grooves or ridges in a barrel of the syringe can be present. Referring to FIG. 3, a barrel 300 is shown that comprises grooves 310, 320, 330, 340 and 350 spaced apart a desired distance along the longitudinal axis of the barrel 300. The distance spacing between the grooves310-350 can be selected to determine how much volume is dispensed as a radial projection moves from one of the grooves 310-350 to another of the grooves 310-350. In some instances, the distance is selected such that movement from one groove to the next results in dispensing about 0.1 cc to about 10 cc, more particularly about 0.5 cc to about 5 cc, for example, about 1 cc to about 3 cc. In certain embodiments, a plunger with a single radial projection can be used with the barrel 300. In use, the plunger is coupled to the barrel by placing a distal end of the plunger into the top surface 301 of the barrel 300. Prior to placement, the barrel can be filled with a liquid comprising an agent such as botulinum toxin. Alternatively, the plunger may be placed fully into the barrel and retracted to draw fluid into the barrel 300. Once the agent is loaded into the barrel 300, the plunger can be depressed until the radial projection engages the groove 310. Successive depressions of the plunger move the radial projection to sequentially engage grooves 320-350. As the plunger projection engages each groove, a tactile feedback force is communicated to the user indicating a metered volume has been dispensed into a particular injection site. The user may then move the needle to another injection site prior to further depression of the plunger. This process may be repeated until a desired number of injections are performed or all fluid in the barrel 300 is dispensed. While the barrel 300 is shown as comprising a plurality of grooves, a plurality of ridges may instead be present, e.g., a plurality of projections that project into the interior space between the walls 304 of the barrel 300 may be present. Examples of a barrel including ridges are described in more detail below.

In other configurations, the circumferential grooves may extend about 360 degrees around the barrel such that the radial projection of the plunger will always contact a circumferential grooves as the plunger is depressed. For example, a series of circumferential grooves each positioned in a selected radial plane may be present such that rotation of the plunger during depression will still result in contact with an adjacent groove. In this configuration, precision can be increased to ensure that substantially the same metered volume is delivered as the plunger is depressed from groove to groove.

In some embodiments, the plunger may comprise more than a single radial projection. Referring to FIG. 4A, a plunger 400 is shown that comprises two radial projections 410, 420 spaced about 180 degrees apart from each other. In this configuration, one or more grooves or ridges (at one or more selected number of radial planes) would be present in the barrel (not shown) or a single circumferential groove or ridge that runs about 360 degree in a radial plane of the barrel can be present. For example, two circumferential grooves or ridges of the barrel can be spaced a similar number of degrees apart as the spacing between the projections 410, 420. Referring to FIG. 4B, a barrel 450 is shown that comprises grooves 460 in a first radial plane, grooves 470 in a second radial plane, grooves 470 in a third radial plane, grooves 475 in a fourth radial plane and grooves 480 in a fifth radial plane.

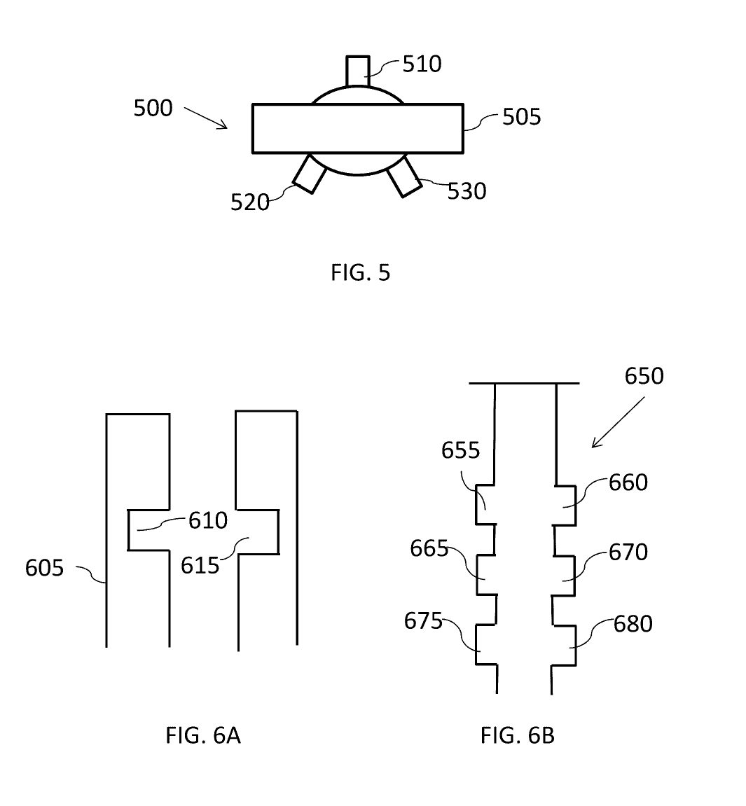

In certain examples, it may be desirable to configure the plunger with three radial projections spaced about 120 degrees apart from each other. A top view of the plunger with three radial projections is shown in FIG. 5. The projections 510, 520 and 530 are spaced about 120 degrees apart and extend outward, e.g., radially, from a longitudinal body. In some instances, the radial projections 510-530 extend substantially orthogonal from the body of the plunger 500, whereas in other examples the radial projections 510-530 may be angled upward or downward relative to the longitudinal axis of the plunger body. The plunger 500 can be inserted into a barrel with at least one ridge or groove in a radial plane, e.g., one, two, three or more circumferential ridges or grooves spaced about 120 degrees apart may be present in each radial plane where the ridges engage a respective one of the projections 510-530. Alternatively, a circumferential groove or ridge that traverses 360 degrees in a radial plane can engage the three projections 510-530. Depression of the plunger 500 by a downward force applied to the top 505 of the plunger 500 results in downward movement of the projections 510, 520 and 530.Where the barrel is configured with a plurality of radial planes each including three or more circumferential grooves or ridges, tactile feedback is provided as the projections 510, 520 and 530 engage the ridges or grooves. By including three projections and three respective ridges or grooves at each of a selected number of radial planes, additional tactile feedback is provided to the user. While the exact number of projections, grooves and ridges present may vary, in some configurations enough projections are present to provide sufficient tactile feedback but not so many projections are present to require undue force to depress the plunger.

In certain configurations, the number of radial planes with grooves or ridges in the barrel may be the same as a desired number of injections to be administered. For example, where a syringe is designed to hold enough fluid for 20 individual injections, the syringe may comprise at least 20 ridges or grooves such that 20 or more metered volumes can be dispensed using the syringe. The particular spacing between ridges or grooves can be selected to match that of a desired metered volume. For example, where depression of a plunger to move the plunger from groove 460 to groove 465 is designed to dispense about 1 cc of volume, the distance between the grooves 460, 465 can be about 1.27 cm where the barrel has a diameter of about 1 cm. Where the syringe is designed to dispense about 0.5 cc as the radial projections of the plunger move from grooves 460 to 465, the distance between the grooves 460, 465 can be about 0.64 cm where the diameter of the barrel is about 1 cm. In certain instances, the spacing between all grooves or ridges along the longitudinal axis is about the same such that the volume dispensed as a result of plunger radial projection movement from ridge to ridge (or groove to groove) is substantially the same. In some configurations, the barrel may comprise about ten to about thirty radial planes with a circumferential ridge or groove, more particularly about fifteen to about twenty-five radial planes with a circumferential ridge or groove, e.g., about 17-22 radial planes with circumferential ridges or grooves.

In certain embodiments, a syringe may comprise a barrel with a groove or ridge in only a single radial plane, and the plunger may comprise a plurality of radial planes each including one or more radial projections. Referring to FIGS. 6A and 6B, a barrel 605 (see FIG. 6A) comprising two grooves 610, 620 in a common radial plane is shown next to a plunger 650 (see FIG. 6B) comprising a first series of projections 655, 660 in a first radial plane, a second series of projections 665, 670 in a second radial plane and a third series of projections 675, 680 in a third radial plane. In use of the syringe, liquid comprising an agent can be loaded into the barrel, and the plunger 650 is inserted into the barrel until the projections 675, 680engage the ridges 610, 615. Alternatively, liquid can be drawn into the barrel 605 by placing the plunger 650 fully into the barrel 605 and retracting the plunger 650 until the projections 675, 680 engage the grooves 610, 615. Depression of the plunger 650 causes the projections 675, 680 to disengage the grooves 610, 615 and move down the barrel 605. After depression a certain amount, the projections 665, 670 engage the grooves 610, 615 to provide tactile feedback to the operator of the syringe and provide an indication that a metered volume of fluid has been dispense from the syringe. The user may then depress the plunger 650 further until the projections 655, 660 engage the grooves 610, 615. This process can be repeated depending on the number of projections present on the plunger and the desired number of injections. While two radial projections in each radial plane are shown as present in the plunger 650, three or more radial projections in each of a selected number of radial planes can be present if desired, e.g., three radial projections may be present in one or more radial planes.

In some configurations, the number of radial planes with two or more radial projections can vary depending on the desired total dispense volume. For example, where it is desirable to inject twenty individual 1 cc injections, the plunger may comprise twenty or more radial planes each comprising two or more radial projections. In some embodiments, radial planes with projections may be spaced apart a desired distance to provide a desired dispense volume as the radial plane is moved downward under the force of a user. For example, the distance between adjacent radial planes can be about 1.27 cm where the barrel has a diameter of about 1 cm. Where the syringe is designed to dispense about 0.5 cc as the radial projections of the plunger move downward, the distance between radial planes of the plunger can be about 0.64 cm where the diameter of the barrel is about 1 cm. In certain instances, the spacing between all radial planes of the plunger along the longitudinal axis of the plunger is about the same such that the volume dispensed as a result of plunger projection movement downward is substantially the same. In some configurations, the plunger may comprise about ten to about thirty radial planes with at least one, two or three radial projections, more particularly about ten to about twenty or about fifteen to about twenty-five radial planes with at least one, two or three radial projections, e.g., about 17-22 radial planes with at least one, two or three radial projections.

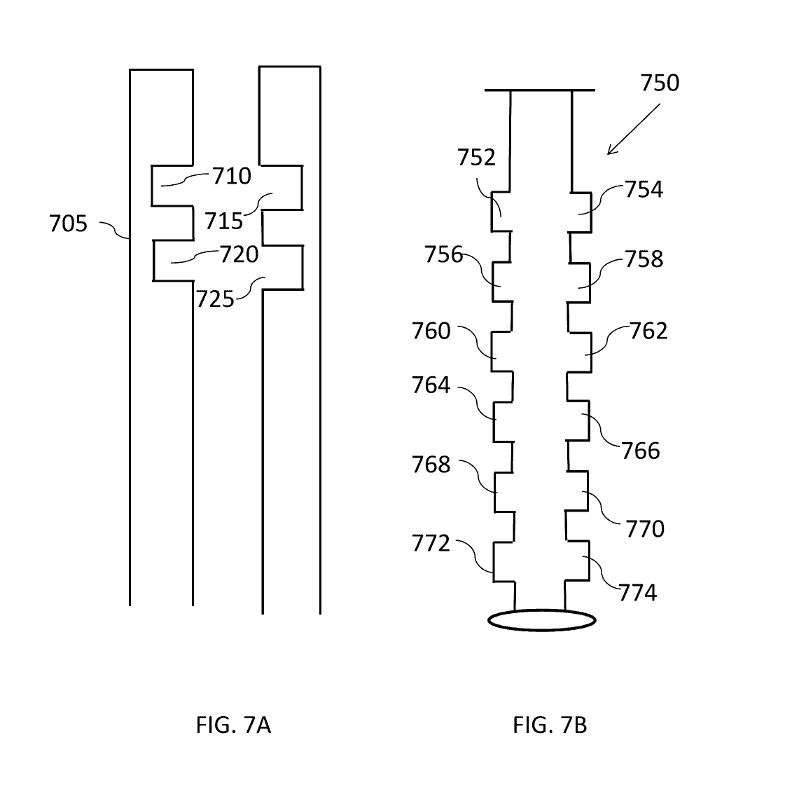

In certain examples, a syringe may comprise a barrel comprising two or more radial planes each comprising a circumferential groove and a plunger comprising two or more radial planes each comprising at least one radial projection. In this configuration, the radial projections in different radial planes may simultaneously engage different grooves of different radial planes to provide for enhanced tactile feedback. In some instances, the grooves can be positioned toward a proximal end of the barrel such that radial projections positioned at a terminal end of the plunger initially engage the grooves during depression. Once the radial projections at the terminal end are pushed past the grooves, then the radial projections slide along the inner wall of the barrel but do not engage any grooves in the barrel. In this manner, enhanced tactile feedback can be provided without requiring too much force to depress the plunger as the plunger is successively depressed. Referring to FIG. 7A, a barrel 705 is shown as comprising grooves 710, 715 in a first radial plane and grooves 720, 725 in a second radial plane. A plunger 750 (see FIG. 7B) comprises a plurality of radial projection pairs 752-774 in six different radial planes. In use of the barrel 705 and the plunger 750, the plunger 750 is inserted into the barrel 705 until the projections 772, 774 engage the grooves 710, 715. This engagement provides tactile feedback to the user. Further depression of the plunger induces engagement of the grooves 710, 715 with the projections 768, 770 and engagement of the grooves 720, 725 with the projections 772, 774. After this engagement occurs, tactile feedback alerts the user that a metered volume of fluid has been dispensed. The user may then move the needle to a different injection site prior to further depression of the plunger 750. Once the needle is appropriately positioned, the plunger 750 can be depressed further until the projections 768, 770 engage the grooves 720, 725 and the projections 764, 766 engage the grooves 710, 715. The projections 772, 774 do not engage any grooves and may be positioned adjacent to the inner wall of the barrel 705 below the grooves 720, 725. After this engagement occurs, tactile feedback alerts the user that a metered volume of fluid has been dispensed into the other injection site. This process can be repeated until a desired number of injections have been performed. By including a series of grooves only at a proximal end of the barrel, suitable tactile feedback can be provided without requiring variable force as the plunger 750 is depressed further into the barrel 705.

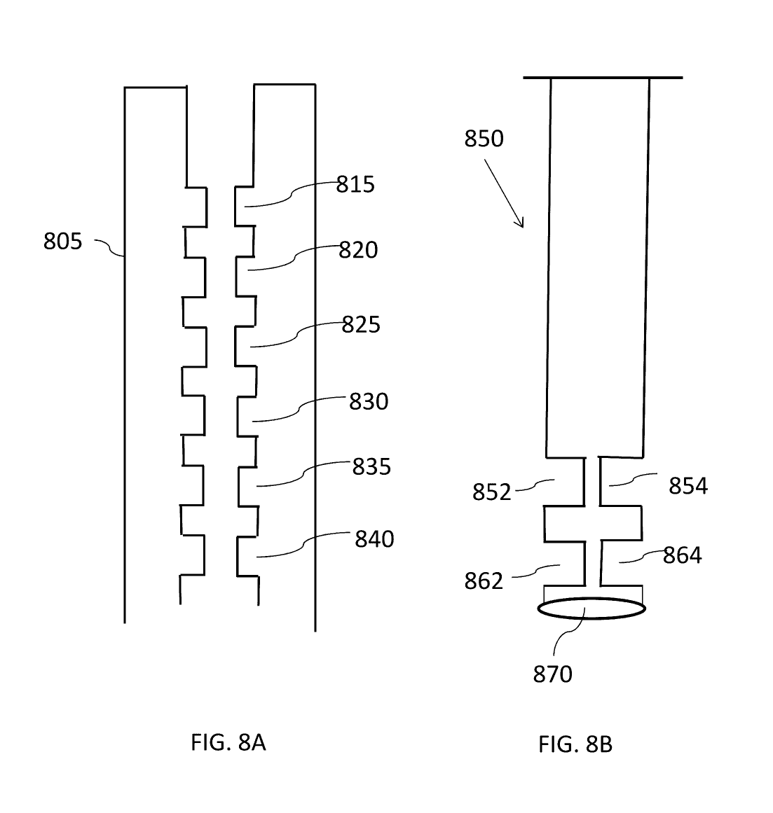

In certain embodiments, the radial projections present in the syringe may instead be present on an inner surface of the barrel and one or more grooves can be present on the plunger. A groove can be distinguished from a projection, for example, in that the projections are configured to flex to permit engagement and disengagement from the grooves, whereas the grooves generally do not bend or move to any substantial degree. Referring to FIGS. 8A and 8B, a barrel 805 includes a plurality of radial planes 815-840 each comprising a set of radial projections designed to engage and disengage a corresponding set of grooves, e.g., either grooves 852, 854 or grooves 862, 864. During use of the syringe, the plunger 850 is inserted into the barrel at a proximal end. The projections in the radial plane 815 engage the grooves 862, 864 to provide tactile feedback. During depression of the plunger 850, the projections at radial plane 815 release and permit downward movement of the plunger 850. At a second position, the radial projections in the radial plane 815 engage the grooves 852, 854, and the radial projections in the radial plane 820 engage the grooves 862, 864. The provision of tactile feedback as the projections and grooves are engaged alert the user that a metered volume has been dispensed into an injection site. Further movement of the plunger 850 down the barrel 805 causes engagement and disengagement of various radial projections of the barrel with the grooves 852, 854, 862 and 864 of the plunger. Once a terminal portion 870 of the plunger reaches the bottom of the barrel 805, e.g., the distal end of the barrel 805, further depression of the plunger 850 is prevented.

The barrels shown in FIGS. 1-7A may include a similar number of radial projections, e.g., ridges, that replace the number of grooves shown. Similarly, the plungers shown in FIG. 1-7B may include a similar number of grooves rather than the number of projections shown in each of those figures. In some instances, the barrel may comprise at least one radial projection in two or more selected radial planes, and the plunger may comprise at least one circumferential groove in a selected radial plane. In certain configurations, the barrel may comprise about the same number of radial planes comprising ridges as the desired number of injections, e.g., 10-25, 15-25 or 15-20, and the plunger may comprise one or more circumferential grooves (or radial projections) that can be engaged and disengaged by the ridges of the barrel.

In certain configurations, the barrel may comprise a circumferential ridge extending outward from the inner surface of the barrel into space intended to be occupied by the plunger in use of the syringe. This circumferential ridge may reversibly engage one or more radial projections of the plunger. The circumferential ridge of the barrel can be positioned proximal, e.g., at an end where the plunger is inserted, so that the ridge does not contact the agent to be injected. In instances where an amino acid based agent is injected, e.g., Botox, a monoclonal antibody, a polyclonal antibody, etc. is injected, it may be desirable to use a smooth walled barrel for the portion of the barrel which will be in contact with the agent to avoid disruption or inactivation of the agent. Where a barrel includes ridges, projections or grooves that could contact the agent, turbulence might be created during plunger depression which could denature or inactivate the agent. By including suitable features in only certain areas of the barrel or the plunger, or both, agent inactivation can be avoided or reduced.

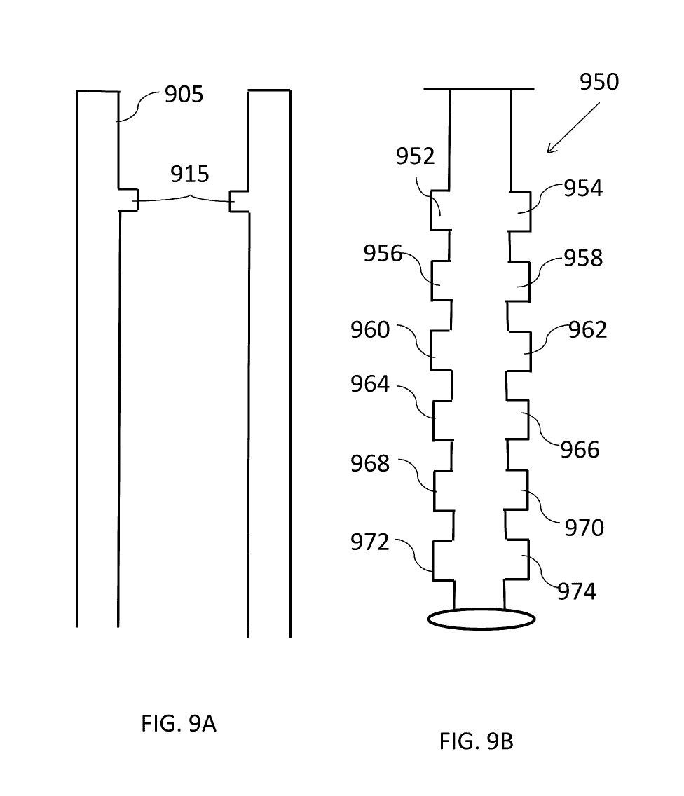

In certain embodiments and referring to FIG. 9A, a barrel 905 is shown comprising a single circumferential ridge 915 that extends into the inner space of the barrel. A plunger 950 (see FIG. 9B) comprising a plurality of radial projections 952-974 can be inserted into the barrel 905. Initially, the projections 972, 974 contact the ridge 915 to provide tactile feedback. Continued depression of the plunger forces the projections 972, 974 downward and past the ridge 915 until the projections 968, 970 contact a top surface of the ridge 915. This movement also results in dispensing of a metered volume of fluid from the syringe. This process can be repeated sequentially until each of the projections is pushed past the ridge 915. The exact number of radial projections on the plunger may vary, and in certain instances the number of radial projections matches the number of metered volume injections desired.

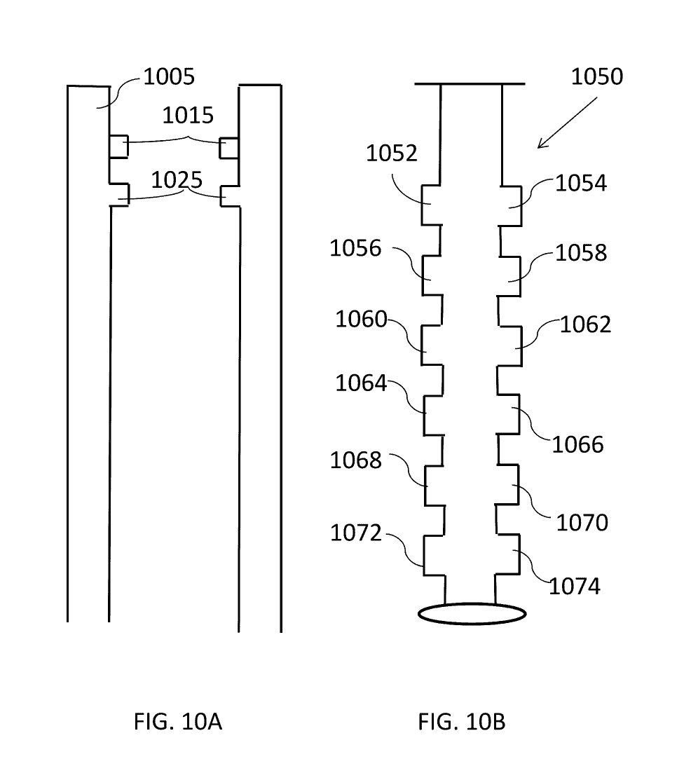

In some configurations, the ridge 915 may be circumferential and comprise one or more projections into the inner surface of the barrel. In some instances, there may be a single ridge which circumferentially spans the entire inner surface of the barrel, whereas in other instances, the ridge may be split into two or more discontinuous sections, e.g., two or more sections spaced about100-180 degrees apart. The exact number of ridges present in the barrel can vary. For example, it may be desirable to have two ridges in two different radial planes to increase the tactile feedback provided by the syringe. One illustration is shown in FIGS. 10A and 10B. A barrel 1005 comprises sets of ridges 1015, 1025 positioned in different radial planes. A plunger 1050 (see FIG. 10B) comprises a plurality of radial projections 1052-1074 that can engage and disengage the ridges 1015, 1025. In use of the barrel 1005 and the plunger 1050, the plunger 1050 can be inserted into the barrel at a proximal end until the projections 1072, 1074 engage the ridges 1015. Additional force can be provided until the projections 1072, 1074 disengage the ridges 1015. Continued depression of the plunger causes projections 1074, 1074 to engage the ridges 1025 and the projections 1068, 1070 to engage the ridges 1015. This process can be repeated until a desired number of metered injections have occurred. In the configurations shown in FIGS. 9A-10B, the portion of the barrel that contacts liquid with agent is smooth, e.g., comprises no ridges or grooves. This smooth wall configuration may be desirable to disrupt agent and to avoid turbulence or bubbling.

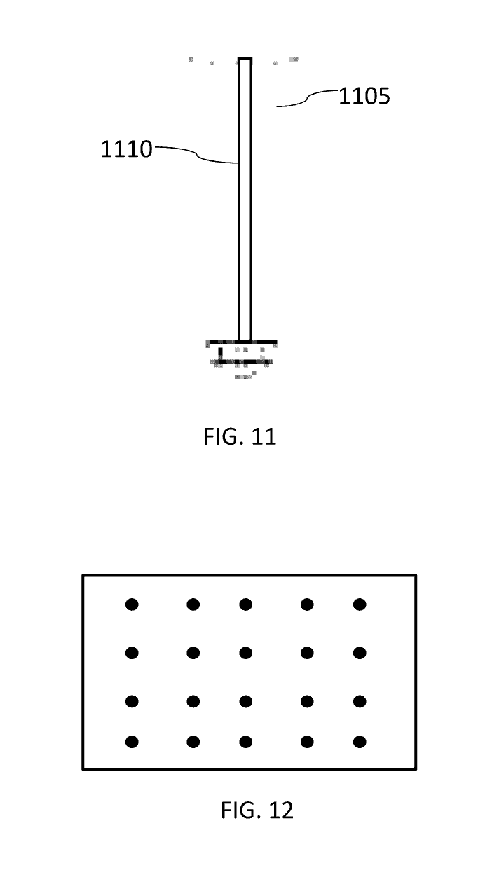

In certain embodiments, where the plunger comprise radial projections, the radial projections may be positioned on one or more longitudinal ribs which may span the length of the plunger. For example, a plunger with longitudinal members running from the top of the plunger to the bottom of the plunger may comprise individual radial projections which can function to engage and disengage the grooves or ridges of the barrel. A simplistic illustration is shown in FIG. 11. The plunger 1100 comprises a longitudinal member 1110 on the body 1105 of the plunger 1100. If desired, the plunger body 1105 may comprise one, two, three, four, five or more longitudinal members, which may be integral to the body. For example, three longitudinal member positioned about 120 degrees apart may be present. One or more of the longitudinal members may engage and disengage a ridge on the barrel to provide the tactile feedback, e.g., a barrel with ridges similar to the ones shown in FIGS. 9A and 10A. Where two or more longitudinal members comprise radial projections, the radial projections on different members can engage different areas of the members for additional tactile feedback.

In certain configurations, the illustrative syringes shown in FIGS. 1-11 can be configured to provide the tactile feedback without providing any audible cue or noise (at a typical distance between a patient and the syringe or a user and the syringe during a treatment procedure) during depression of the plunger. For example, certain syringes may provide an audible noise as the plunger is depressed due to friction produced from movement of the plunger. This audible noise can be concerning to a patient, particularly a patient positioned in stirrups for injection of a botulinum toxin into their bladder wall through a needle inserted into the vagina. By providing tactile feedback without providing any audible feedback, patient anxiety can be reduced. To avoid audible feedback, the projections and/or grooves may comprise one or more elastomeric materials which are effective to resist movement when engaged (to provide the tactile feedback) but do not produce sound waves during depression of the plunger. In other instances, the radial projections of the plunger (or barrel) can be designed to be sufficiently thin to permit some tactile feedback without inducing any audible noise during movement. In further instances, the projections and/or grooves can be coated with a material, e.g., polytetrafluoroethylene, carbon fiber, etc. to increase overall slickness or slipperiness to permit the projections and grooves to engage and disengage without any generation of substantial noises. In yet other instances, the walls of the barrel, e.g., internal wall or external wall or both, can be coated with a sound absorbing material to prevent any sound waves that are produced from exiting the syringe and disturbing the patient. In some configurations, substantially no noise or zero noise is produced as the plunger is depressed even though tactile feedback remains.

In some instances, an internal stop may be present at the bottom of the syringe such that movement of the plunger from the last radial projection to the stop prevents further injections using the syringe. The internal stop can be designed to prevent injection of too large a fluid volume as a terminal end of the plunger approaches a distal end of the barrel. In other instances, the terminal or distal end of the barrel can be designed to engage a terminal or distal end of the plunger to prevent further depression of the plunger.

In other examples, at least one of the circumferential ridges is configured to permit one way movement of the plunger in the barrel. For example, the circumferential ridges can be designed asymmetrically to permit engagement and release of the radial projections when the plunger is depressed but capture the radial projections if the plunger is attempted to be retracted. In other instances, each of the circumferential ridges is configured to permit one way movement of the plunger in the barrel. In some embodiments, at least one of the radial projections of the plunger is configured to permit one way movement of the plunger in the barrel. In certain embodiments, each of the radial projections of the plunger is configured to permit one way movement of the plunger in the barrel. Configurations that are designed for one way movement permit insertion of the plunger into the top of the syringe and capturing of the plunger by the barrel once the plunger has been depressed. Such configurations prohibit reuse of the syringe to avoid any contamination issues.

In certain examples, the syringes described herein can be used to inject a plurality of injection sites in a bladder wall to treat overactive bladder. While the exact agent used may vary, in some embodiments the agent comprises a botulinum toxin or is a botulinum toxin. Illustrative botulinum toxins include, but are not limited to, type A, A1, A2, B, C, C1, D, E, F or G botulinum toxins. In some instances, the syringe is filled with a solution comprising onabotulinumtoxinA (Botox® material), which is commercially available from Allergen. While not wishing to be bound by any particular theory, the botulinum toxin injected into the bladder wall can block nerve signals from reaching the muscles of the bladder wall. This blockage can reduce the urgent need to void. If desired, a combination of different types of botulinum toxin can be injected. In other instances, a therapeutic agent may be co-administered with the botulinum toxin using the syringes described herein. For example, an anti-neoplastic agent can be co-administered with the botulinum toxin to treat cancerous areas of the bladder near the bladder wall and treat the overactive bladder. In some examples, a drug used to treat overactive bladder may be administered using one of the syringes described herein or co-administered with the botulinum toxin using one of the syringes described herein. Illustrative drugs include, but are not limited to, darifenacin, fesoterodine, mirabegron, oxybutynin, solifenacin, tolterodine and trospium. Co-administration of a drug to treat overactive bladder with botulinum toxin can inhibit different pathways to treat the overactive bladder.

In certain examples, to treat the overactive bladder with a botulinum toxin, a needle is coupled to one of the syringes described herein. A cystoscope or other imaging device may then be inserted into the vagina to provide visualization of the bladder wall. The needle can then be inserted into the vagina and positioned adjacent to a first site on the bladder wall. The user may then press the needle into a first site of the bladder wall and depress the plunger from a first position to a second position to inject a metered volume into the first injection site. Tactile feedback from the syringe alerts the user that the desired metered volume has been injected into the site. The user may then move the needle to one or more additional sites in the bladder wall and perform an injection in a similar manner. The grooves and projections present in the syringe function as “internal stops” in that the user is alerted to stop injection into a particular site once tactile feedback is provided from engagement of the projections and the grooves. This process may be repeated a desired number of times until a sufficient number of independent injections into the bladder wall are provided to treat the overactive bladder, e.g., 10-25 or 15-25 injections of a botulinum toxin into different sites of the bladder wall.

In certain instances, the botulinum toxin can be injected in a grid pattern in the bladder wall. Referring to FIG. 12, an injection grid comprising about four rows and five columns is shown that can be used to provide about 20 independent injections into the bladder wall. Where fewer or more injections are desired, the number of rows and columns may be less or more, respectively. In some instances, a user injects about 2-8 different rows of injections and about 3-9 different columns of injections, e.g., an array of 2-8 rows by 3-9 columns may be used to provide the injections. To facilitate injection, a grid overlay can be provided on a video screen, e.g., a video screen of a cystoscope or a mobile device wirelessly coupled to a sensor in the cystoscope, to assist the user in the injection process. For example, a virtual grid overlay can be digitally provided on the screen to guide the injections. The grid may be calibrated to a reference position and include a motion sensor that can adjust the grid positions based on movement of the camera or cystoscope. If desired, the user may touch the particular grid site on the screen after injection at that particular site to provide guidance about additional injection sites that remain and to avoid duplicate injection into any site. As the user presses the grid site, the particular “dot” on the grid site can be removed leaving behind the remaining dots each of which represents individual injection sites. In addition, while the rows and columns in FIG. 12 are shown as being aligned with each other, different rows or different columns can be offset by a desired amount of spacing if desired. The injection sites may also be asymmetric in that more injection sites may be present in one area of the bladder wall than another area.

In certain embodiments, one or more of the syringes described herein can be used to inject each of the plurality or injections into the bladder wall. In some examples, the entire injection process may be performed without being able to view the syringe during the injection procedure. For example, the tactile feedback that results from engagement of the radial projections and grooves present within the syringe permits the entire procedure to be performed even where the body of the syringe is occluded from visualization by the user. In some instances, fifteen or twenty individual injections can be made in the bladder wall within an injection period, e.g., 5 minutes or less or 10 minutes or less.

In certain examples, various sizes of syringes can be used in the injection process. To avoid contamination and/or reuse of the syringe, a syringe can be selected to provide a desired overall volume level. For example, where ten injections are desired with 1 cc per injection, the syringe can be sized and arranged with enough grooves and projections to permit the ten injections to be accomplished with tactile feedback. In some instances, the syringe may be sized to provide a total volume of at least 15 cc or at least 20 cc or more.

In some embodiments, a counter or counting device may be used with the syringe such that depression of the plunger from one groove to an adjacent groove results in a count or counter movement on a screen to provide some feedback to the user about how many injections have been performed. The counting device may be part of a larger system that is used with the syringe to provide a visual grid used in the injection procedure.

In certain embodiments, injection of the bladder wall with a botulinum toxin can result in treatment of the overactive bladder. To the extent the injections reduce the sensation to void, a bladder training device, e.g., mobile device, watch, etc., along with a bladder training method can optionally be used to train the bladder to void at desired intervals. For example, the bladder training device can be configured to generate an audible alarm at desired intervals to notify the wearer to void. In continuous use of the bladder training device, the bladder may be able to learn or be trained to provide voiding sensations at desired intervals. In some instances, the bladder training may take the form of a software application which resides on a patient's mobile device. The software application may remind the subject to void at selected intervals and may require the subject to acknowledge voiding has taken place by generating an audible alarm until the subject presses a button or key of the mobile device. The combination of treating overactive bladder with botulinum toxin injections and training of the bladder using a voiding schedule may reduce the need for successive botulinum toxin treatments or may permit reduction in the amount of botulinum toxin that is injected into each site. The software application may also be designed to initiate prompts to perform Kegel exercises (or other muscle strengthening exercises such as those which can strengthen the pubococcygeus muscle or other pelvic floor structures) at selected intervals to further enhance treatment of the overactive bladder. The mobile device typically takes the form of a cellular phone, but it may also be configured as a tablet, computer, watch that works with a mobile device, e.g., Apple Watch, a wearable device such as Google® Glass or other mobile devices which include a processor and an optional touch screen and operating system. The mobile device also may include one or more memory units which can store the software application and permit access by the processor.

In some examples, the injection procedure may involve removal of a syringe already pre-loaded with a botulinum toxin, e.g., pre-loaded with lyophilized botulinum toxin. The user can reconstitute the botulinum toxin to a desired concentration by withdrawing a buffer or injection fluid into the syringe by retracting the plunger until a suitable volume is present. Volume indicia, e.g., volume markings at desired intervals, can be present so the user can verify the appropriate volume has been drawn into the syringe. To avoid degradation of the toxin, the pre-loaded syringe may be refrigerated or kept cool prior to use. In certain instances, the syringe may be agitated to induce the botulinum toxin to dissolve or be suspended in the injection solution. Once dissolved, the injection procedure can be initiated.

In certain embodiments, the syringes described herein can be packaged in a kit with instructions for using the syringe to treat overactive bladder. For example, the instructions may specify the number of injections, injection volume, etc. that can be used to treat overactive bladder. The kit may also comprise one or more botulinum toxins for use in treating the overactive bladder, e.g., the kit may comprise onabotulinumtoxinA. In some instances, the botulinum toxin may be packaged in a vial separate from the syringe or can be packaged in the syringe itself. In certain examples, one or more bladder treatment drugs may be present in the kit. If desired, an injection solution effective to solubilize the lyophilized botulinum toxin may be present in the kit. In some instances, the kit may comprise a needle configured to couple to the syringe. In certain examples, the kit may comprise a fluid line configured to couple to the needle and to the syringe.

In certain examples, a method of treating overactive bladder can include providing a syringe as described herein, and providing instructions for using the syringe with a botulinum toxin to treat overactive bladder. In some instances, the method comprises providing the botulinum toxin. In other examples, the method comprises providing a needle configured for use with the syringe. In further embodiments, the method comprises providing a training device or training app configured to retrain the treated overactive bladder. In certain examples, the method comprises providing instructions for retraining the treated overactive bladder.

In some configurations, the syringes can be used to introduce fluids, agent, toxins, etc. into subject with the assistance of one or more imaging techniques. For example, ultrasound imaging, magnetic resonance imaging or thermal imaging can be used to guide a needle coupled to the syringe to a desired area or anatomical site. In one instance, ultrasound imaging may be used to guide a needle transvaginally to a site adjacent to the bladder to permit injection of agent near the site, e.g., injection of a vesicular agent such as vesicular botulinum toxin. Transvaginal injection can reduce the occurrence of urinary tract infections as the needle is not inserted through the urethra. If desired, transurethral injection may be used in combination with transvaginal injection to enhance treatment of overactive bladder.

While the syringes and their use have been described in various instances in reference to treating overactive bladder, the syringes may also be used to introduce fluids into a patient in procedures other than urological procedures. For example, the syringes can be used to introduce a metered volume of a fluid or dye into a vein or artery during radiology or cardiology procedures. A desired amount of drug or agent can be introduced into the colon during a colonoscopy procedure where it may not be possible to view the syringe during the procedure.

Certain specific configurations of a syringe are described below to further illustrate the syringes and their use.

### EXAMPLE 1

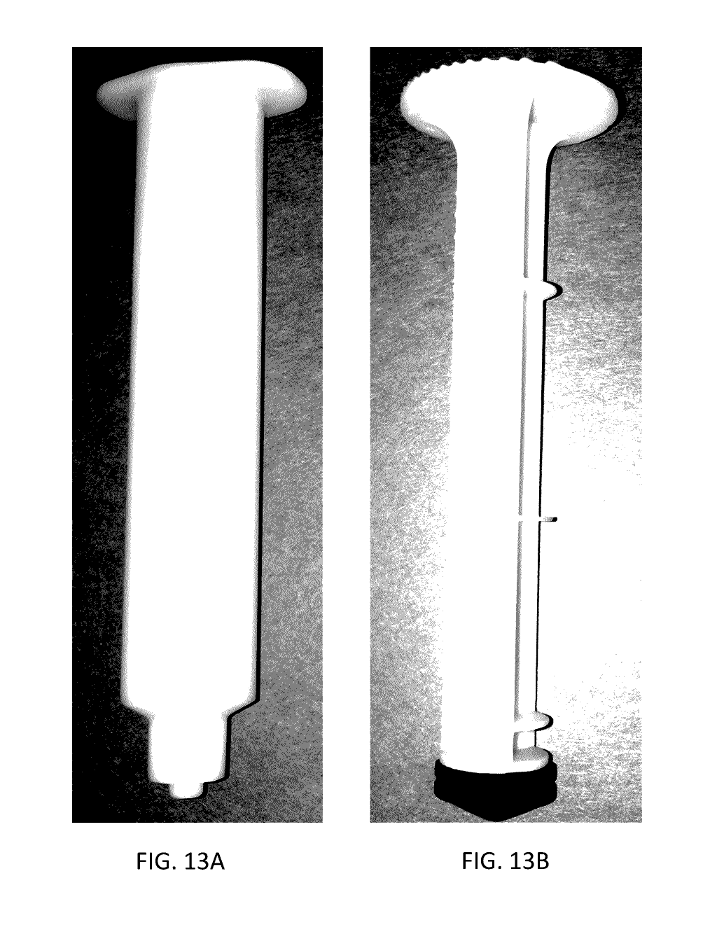

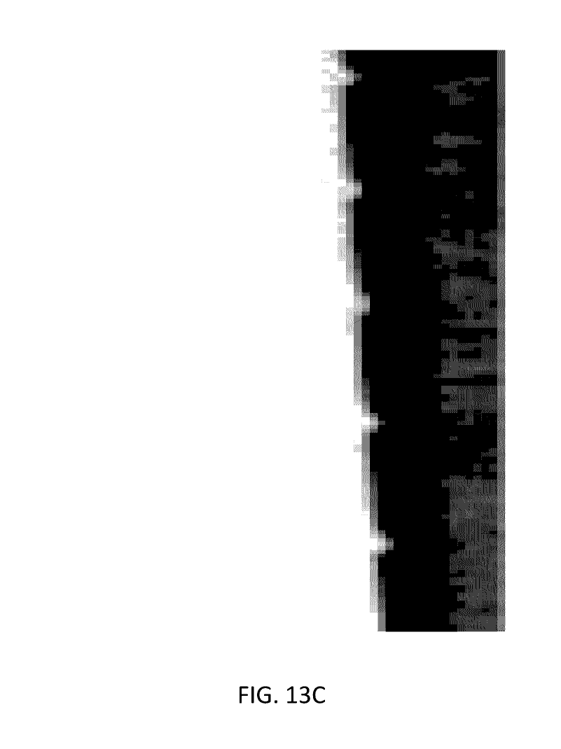

As shown in the photographs of FIGS. 13A-13C, a barrel (FIG. 13A) and plunger (FIG. 13B) can be used together to provide a tactile feedback syringe. The plunger comprises three longitudinal fins each comprising a plurality of projections (see FIG. 13C for an enlarged view). The barrel comprises an internal ridge that engages the projections of the plunger. The projections of different longitudinal fins can be spaced within the same radial plane such that different projections in the same plane engage the ridge of the barrel at the same time to provide for tactile feedback.

When introducing elements of the examples disclosed herein, the articles “a,” “an,” “the” and “said” are intended to mean that there are one or more of the elements. The terms “comprising,” “including” and “having” are intended to be open-ended and mean that there may be additional elements other than the listed elements. It will be recognized by the person of ordinary skill in the art, given the benefit of this disclosure, that various components of the examples can be interchanged or substituted with various components in other examples.

Although certain aspects, examples and embodiments have been described above, it will be recognized by the person of ordinary skill in the art, given the benefit of this disclosure, that additions, substitutions, modifications, and alterations of the disclosed illustrative aspects, examples and embodiments are possible.

1. A method of treating overactive bladder, the method comprising injecting a bladder wall at a plurality of individual sites with a botulinum toxin from a syringe configured with a plurality of individual internal stops to dispense a defined volume from the syringe at each injection site as a plunger of the syringe is depressed from a first internal stop to a second internal stop to treat the overactive bladder.

2. The method of claim 1, further comprising injecting the botulinum toxin in a mxn array into the bladder wall.

3. The method of claim 2, in which m of the array is two to twenty and n of the array is two to twenty.

4. The method of claim 1, further comprising configuring the syringe to provide tactile feedback at each of the plurality of individual internal stops.

5. The method of claim 4, further comprising configuring the syringe to provide substantially no noise as the plunger is depressed from the first stop to the second stop.

6. The method of claim 1, further comprising configuring the syringe with a total volume to permit injection into at least twenty individual sites in the bladder wall.

7. The method of claim 1, further comprising configuring a barrel of the syringe with an interior ridge that engages a projection on a plunger of the syringe at the first internal stop.

8. The method of claim 1, further comprising loading the syringe with lyophilized botulinum toxin.

9. The method of claim 1, further comprising solubilizing the lyophilized botulinum toxin by introducing a liquid into the syringe with the lyophilized botulinum toxin.

10. The method of claim 1, further comprising selecting the botulinum toxin to be onabotulinumtoxinA.

11. The method of claim 1, further comprising positioning the syringe to inject substantially the same amount of botulinum toxin at each of the plurality of individual sites in the bladder wall when a user injecting the botulinum toxin cannot view the syringe.

12. The method of claim 1, further comprising using the syringe with a cystoscope to inject the botulinum toxin.

13. The method of claim 12, further comprising using a device configured to count the number of injections into the bladder wall.

14. The method of claim 12, further comprising using a device configured with a processor during the injection method.

15. The method of claim 14, further comprising configuring the processor to display a grid on an image provided by the cystoscope.

16. The method of claim 15, further comprising configuring the grid with an indicator site of each of the plurality of injection sites and removing the indicator once the syringe has been used to inject the botulinum toxin into the bladder wall at a particular indicator site.

17. The method of claim 1, further comprising using the syringe to inject at least twenty individual sites in 10-25% less time than when using a syringe missing the first internal stop and the second internal stop.

18. The method of claim 1, further comprising configuring a plunger of the syringe with a plurality of radial projections and configuring a barrel of the syringe with a plurality of interior ridges configured to provide tactile feedback when at least one of the radial projections contacts at least one of the interior ridges.

19. The method of claim 18, further comprising configuring the plunger to comprise three radial projections spaced about 120 degrees apart and configuring the barrel with a plurality of circumferential interior ridges spaced longitudinally along the barrel, in which depression of the plunger from a first longitudinally spaced circumferential ridge to a second longitudinally spaced circumferential ridge dispenses a metered volume of the botulinum toxin from the syringe.

20. The method of claim 1, further comprising training a bladder muscle by inducing voiding of the bladder at a selected interval.

21-55. (canceled)
